(19) United States
(12) Patent Application Publication
(10) Pub. No.: US 2023/0309595 A1
(43) Pub. Date: Oct. 5, 2023

(54) DIETARY SUPPLEMENT COMPOSITIONS COMPRISING GANODERMA AND METHODS FOR MAKING

(71) Applicants: Ricardo Gutierrez, Idaho Falls, ID (US); Melaleuca, Inc., Idaho Falls, ID (US); Melaleuca (China) Wellness Products Co. Ltd., Shanghai (CN); Melaleuca (Nantong) Wellness Science and Technology Co., Ltd., Nantong, Jiangsu (CN)

(72) Inventors: Bruce Bu, Shanghai (CN); Ricardo Gutierrez, Idaho Falls, ID (US); Gerry Ge, Jiangsu (CN)

(21) Appl. No.: 18/023,919

(22) PCT Filed: Aug. 31, 2021
(86) PCT No.: PCT/US2021/048538

§ 371 Date: Feb. 28, 2023

(30) Foreign Application Priority Data
Aug. 31, 2020 (WO) PCT/CN2020/112630

(51) Int. Cl. A23L 31/00; A23L 33/105; A23P 10/20; A23P 10/30; A23P 20/10; A61K 36/074; A61K 9/16

(52) U.S. Cl. CPC A23L 31/00; A23L 33/105; A23P 10/20; A23P 10/30; A23P 20/10; A61K 36/074; A61K 9/167; A61K 9/1688

(57) ABSTRACT

This document provides dietary supplement compositions. For example, this document provides dietary supplement compositions that include a Ganoderma lucidum extract.

### CROSS-REFERENCE TO RELATED APPLICATIONS

This application claims benefit of priority from PCT Application No. PCT/CN2020/112630, filed on Aug. 31, 2020.

### TECHNICAL FIELD

This document relates to the field of dietary supplements. For example, this document relates to methods for making dietary supplement compositions useful for human or animal consumption.

### BACKGROUND INFORMATION

Many people desire improved health and well-being, particularly with their immunity and overall body health. Ganoderma lucidum is an herb that can be used in dietary supplements to support or maintain positive immunity and overall body health. Certain herbal powders, such as Ganoderma lucidum powder, can be produced using a spray drying process. Because some herbal powders (including Ganoderma powders) are very hygroscopic, however, the resultant powders tend to absorb moisture.

### SUMMARY

This document provides dietary supplement compositions and methods for making dietary supplement compositions. For example, this document provides methods for making dietary supplement compositions useful for human or animal consumption. In some cases, the methods described herein can be used to make dietary supplement compositions that include at least one extract, such as a Ganoderma lucidum extract.

In general, one aspect of this document features methods for producing a composition containing at least one extract, such as a Ganoderma lucidum extract. In some cases, a dietary supplement composition can be obtained using a method described herein for producing at least one extract (e.g., a Ganoderma lucidum extract). In some cases, a health care product (e.g., a capsulated product) containing at least one extract (e.g., a Ganoderma lucidum extract) can be obtained using the methods provided herein.

In some aspects, this document features a method of making a composition, where the method includes (1) granulating a powdered first sample of Ganoderma lucidum extract to form granulates, and (2) applying a coating material on the granulates, the coating material containing a second sample of Ganoderma lucidum extract, where the composition is substantially free of non-Ganoderma lucidum derived materials.

In some cases, the granulating can be performed without using non-Ganoderma lucidum derived materials. In some cases, the coating step can be performed without using non-Ganoderma lucidum derived materials. In some cases, the granulates can be substantially free of auxiliary excipients or additives. In some cases, the coating material can be substantially free of auxiliary excipients or additives. In some cases, the powder can be a Ganoderma lucidum extract. In some cases, the granulating step can include dry granulating the powder. In some cases, the applying step can include applying a solution containing a solute and a solvent (e.g., water) to the granulates. In some cases, the solute can be a Ganoderma lucidum extract. The solvent can include non-organic solvents, or organic solvents. In some cases, the solvent can be water. In some cases, the solution can contain about 1% w/w to about 40% w/w of a Ganoderma lucidum extract. In some cases, the composition can include particles having a dimension ranging between a 16 mesh size and a 100 mesh size (or between 0.0469 inch and 0.0059 inch). In some cases, the composition can have a bulk density of about 250 mg/ml to about 2000 mg/ml. In some cases, the coating material can be applied by a bottom-spray fluidized bed. In some cases, the powder can be obtained by subjecting Ganoderma lucidum to water extraction and then spray drying.

In some cases, this document features a composition made using any of the methods described herein.

In some aspects, this document features a health care product, where the health care product includes, as an active ingredient, a composition prepared by any of the methods described herein.

In some aspects, this document features a capsulated product containing, as an active ingredient, a composition prepared by any of the methods described herein.

In some aspects, this document features a method of making a composition, where the method includes granulating a powder containing one or more extracts to form granulates, and applying a coating material to the granulates, where the coating material contains the one or more extracts, and where the composition is substantially free of materials that are not derived from the one or more extracts.

In some cases, the one or more extracts each can have a bulk density less than 200 mg/ml. In some cases, the one or more extracts can include a Ganoderma lucidum extract. In some cases, the composition can have a bulk density of about 250 mg/ml from about 2000 mg/ml.

In some cases, the methods provided herein can be used to make a composition containing about 100 mg to about 800 mg of a Ganoderma lucidum extract. In some cases, the composition can be a solid composition that includes a plurality of particles (e.g., granulates). In some embodiments, the granulates, collectively, in a composition can contain about 100 mg to 600 mg of a Ganoderma lucidum extract. In some embodiments, the methods described herein can be used to make a solid composition that includes a plurality of coated particles (e.g., granulates). In some cases, a coating material in the composition comprises, collectively, about 1 mg to about 50 mg of a Ganoderma lucidum extract. In some cases, the composition can have a moisture content that is reduced compared to the moisture content of a corresponding composition that lacks the coating material. In some cases, the coated granulates can have a porosity that is reduced compared to the porosity of corresponding uncoated granulates.

In some aspects, this document features a method of making a composition, where the method includes (1) granulating a powdered first sample of Ganoderma extract to form granulates, and (2) applying a coating material on the granulates, where the coating material contains a second sample of Ganoderma extract. In some cases, the composition can be substantially free of non-Ganoderma derived materials.

The dietary supplement compositions provided herein can contain one or more extracts (e.g., one or more Ganoderma extracts), which can advantageously boost the immune system, provide anti-cancer properties, provide antioxidant properties, prevent or reduce fatigue and/or depression, and/or benefit the heart health and/or blood sugar levels of a consumer.

The compositions and methods provided herein can have several benefits. In some cases, for example, the methods described herein can be used to advantageously produce a composition containing an extract (e.g., a Ganoderma lucidum extract) having a lower moisture absorption as compared to a spray dried extract powder containing the extract (e.g., a Ganoderma lucidum extract). In particular, in some embodiments, the methods provided herein can be used to produce a composition having coated particles (e.g., granulates) such that the composition exhibits improved moisture resistance. Preventing moisture from entering the composition can improve the production efficiency and shelf life of a dietary supplement composition.

In some cases, the methods provided herein can advantageously be used to produce a composition made of a pure extract that does not include any components that are not derived from that same extract. For example, in some embodiments, the methods provided herein can be used to produce a pure Ganoderma lucidum extract that does not include any non-Ganoderma lucidum ingredients. The compositions provided herein can have an increased amount of an active ingredient (e.g., a Ganoderma lucidum extract) as compared to other products that include non-active ingredients (e.g., non-Ganoderma lucidum substances), such as auxiliary excipients, additives, fillers, and/or process additives. Inclusion of non-active ingredients (e.g., non-Ganoderma lucidum ingredients) can reduce the amount of the active ingredient in the final product.

In some cases, the compositions and methods provided herein can be used to produce a dietary supplement composition with a higher bulk density that is more desirable for efficient manufacturing. An increased bulk density can also increase the amount of active ingredients present in a finished product (e.g., capsulated formulations). A Ganoderma lucidum extract powder can be obtained by spray drying an extract solution. In some cases, however, spray drying can produce a porous and loose powder having a small particle size (e.g., particles size <100 mesh particles, or <0.0059 inches) that can be undesirable. For example, due to the small particle size, the amount of the extract powder (e.g., filling weight) in a finished product (e.g., capsules) can be lower than desired (<450 mg for capsules).

While the embodiments in this document describe compositions containing a Ganoderma lucidum extract and methods of producing compositions containing a Ganoderma lucidum extract, the scope of the inventions described herein is applicable to compositions, and methods for making compositions, containing any extract powder that is hydrophobic, has a low bulk density (e.g., <200 mg/ml), or both. A Ganoderma lucidum extract powder is one example of an extract powder that is hydrophobic and generally available as a powder having a low bulk density (e.g., <200 mg/ml). Furthermore, the compositions provided herein can be made from one or more extract powders, where each of the extract powders is hydrophobic and has a low bulk density (e.g., <200 mg/ml).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a flow chart depicting an exemplary process for producing a dietary supplement composition provided herein.

FIG. 2 is a diagram illustrating an exemplary spray fluidized bed that can be used for coating particles.

FIGS. 3A-3C each contains a series of images of Ganoderma lucidum products at various times, including a powder after spray drying (FIG. 3A), particles resulting from dry granulation (FIG. 3B), and particles resulting from dry granulation and coating (FIG. 3C) with a self-coating of Ganoderma lucidum solution. Images were taken after incubation at 30° C. and 60% relative humidity (RH) for 0, 0.5, 1, 1.5, 2, and 3 hours).

FIG. 4A includes images showing a spray dried Ganoderma lucidum powder (left), dry granulated Ganoderma lucidum particles (center), and dry granulated Ganoderma lucidum particles that were coated with a solution of Ganoderma lucidum powder (right). FIG. 4B includes close-up images of dry granulated (left) and dry granulated and coated (right) Ganoderma lucidum particles, while FIG. 4C includes magnified images of dry granulated (left) and dry granulated and coated (right) Ganoderma lucidum particles.

### DETAILED DESCRIPTION

This document provides dietary supplement compositions that include at least one extract (e.g., a Ganoderma lucidum extract) and methods for making dietary supplement compositions that include the at least one extract (e.g., a Ganoderma lucidum extract). A dietary supplement composition provided herein can be in the form of a liquid, solution, suspension, tablet, powder, cream, mist, atomized vapor, aerosol, soft gelatin capsule, hard gelatin capsule, gel, confectionary, shake, bar, or supplemented food. Any of the embodiments provided herein are generally applicable to compositions containing, and methods used for making compositions containing, at least one extract powder that is hydrophobic, has a low bulk density (e.g., <200 mg/ml), or both. Any examples and embodiments specific to compositions containing a Ganoderma lucidum extract and methods of producing compositions containing a Ganoderma lucidum extract may be applied generally to compositions containing, and methods for making compositions containing, one or more extracts, where each extract is hydrophobic and/or has a low bulk density.

A dietary supplement composition provided herein can contain one or more ingredients derived from a Ganoderma lucidum mushroom (e.g., a Ganoderma lucidum extract). The dietary supplement compositions provided herein can made from materials derived from any portion of a Ganoderma lucidum mushroom. For example, the Ganoderma lucidum content can be made from any portion of the fruit body, such as the stem, cap, and/or spores.

A Ganoderma lucidum component used in making the compositions described herein can be synthesized or derived from natural sources. In some cases, the Ganoderma lucidum contained in the compositions provided herein can be a powder or liquid extract. The extract can be in the form of a liquid (e.g., an aqueous solution), a solid (e.g., a particulate or a powder), or a semi-solid form (e.g., a gel or an emulsion). In some cases, an extract of Ganoderma lucidum can be made by applying a water extraction using purified water or distilled water as a solvent. In some cases, Ganoderma lucidum extracts can be obtained as described elsewhere (see, e.g., Chinese Patent No. CN1813820A). In some cases, Ganoderma lucidum extracts can be obtained commercially. In some cases, an alcohol (e.g., ethanol) extraction or a hydroalcoholic extraction can be employed to obtain an extract of Ganoderma lucidum.

A dietary supplement composition provided herein can contain any appropriate amount of a Ganoderma lucidum extract. In some cases, for example, a dietary supplement composition provided herein can contain between about 10 mg and about 1,000 mg (e.g., between about 100 mg and about 800 mg, between about 200 mg and about 600 mg, between about 400 mg and about 500 mg, between about 100 mg and about 500 mg, between about 200 mg and about 500 mg, or between about 300 mg and about 500 mg) of a Ganoderma lucidum extract. In some cases, a dietary supplement composition provided herein can be formulated to contain an amount of a Ganoderma lucidum extract such that a daily dose of between about 10 mg and about 500 mg (e.g., between about 25 mg and about 500 mg, between about 50 mg and about 500 mg, between about 100 mg and about 500 mg, between about 10 mg and about 400 mg, between about 10 mg and about 300 mg, between about 10 mg and about 200 mg, between about 10 mg and about 150 mg, between about 50 mg and about 150 mg, between about 60 mg and about 140 mg, or between about 75 mg and about 125 mg) of the Ganoderma lucidum extract can be conveniently administered.

The compositions provided herein can include one or more active ingredients. In some cases, a composition provided herein includes only one or more (e.g., one, two, three, four, five, or more than five) active ingredients derived from a Ganoderma lucidum mushroom. An exemplary active ingredient of a Ganoderma lucidum mushroom or portions thereof can be in the form of a Ganoderma lucidum extract.

In some cases, the compositions provided herein can include one or more active ingredients from a Ganoderma species other than Ganoderma lucidum. For example, a composition provided herein can includes one or more (e.g., one, two, three, four, five, or more than five) active ingredients derived from a Ganoderma mushroom. An exemplary active ingredient of a Ganoderma mushroom or portions thereof can be in the form of a Ganoderma extract. An example of a Ganoderma species that can be a source of one or more active ingredients is Ganoderma sinense. Other Ganoderma species also may be used as a source of one or more active ingredients.

A dietary supplement composition can contain any appropriate amount of an active ingredient. For example, 100% or less (e.g., 50% or less, 60% or less, 70% or less, 80% or less, 90% or less, 95% or less, 96% or less, 97% or less, 98% or less, 99% or less, 99.9% or less, 50% or more, 60% or more, 70% or more, 80% or more, 85% or more, 90% or more, 95% or more, 96% or more, 97% or more, 98% or more, or 99% or more) of a dietary supplement composition provided herein can be an active ingredient (e.g., a Ganoderma lucidum extract). In some cases, a dietary supplement composition provided herein can contain between about 10 mg and about 500 mg (e.g., between about 25 mg and about 500 mg, between about 50 mg and about 500 mg, between about 100 mg and about 500 mg, between about 10 mg and about 400 mg, between about 10 mg and about 300 mg, between about 10 mg and about 200 mg, between about 10 mg and about 150 mg, between about 50 mg and about 150 mg, between about 60 mg and about 140 mg, or between about 75 mg and about 125 mg) of an active ingredient (e.g., a Ganoderma lucidum extract). In some cases, a dietary supplement composition provided herein can be formulated to contain an amount of an active ingredient, such that a daily dose includes between about 10 mg and about 500 mg (e.g., between about 25 mg and about 500 mg, between about 50 mg and about 500 mg, between about 100 mg and about 500 mg, between about 10 mg and about 400 mg, between about 10 mg and 300 about mg, between about 10 mg and about 200 mg, between about 10 mg and about 150 mg, between about 50 mg and about 150 mg, between about 60 mg and about 140 mg, or between about 75 mg and about 125 mg) of the active ingredient (e.g., a Ganoderma lucidum extract).

In some cases, a dietary supplement composition provided herein can be a solid composition. In some cases, a solid composition can include particles, where the particles are made only of ingredients derived from an extract or extract source (e.g., Ganoderma lucidum). In some embodiments, a dietary supplement composition provided herein includes particles (e.g., granulates) of a desired dimension. For example, in some cases, the dietary supplement composition provided herein contains particles having a dimension ranging between a 16-mesh size and a 100-mesh size (or between 0.0469 inch and 0.0059 inch). In some cases, the dietary supplement composition provided herein contains particles (e.g., granulates) having a dimension with a mesh size less than a 5-mesh size (e.g., less than a 5-mesh size, less than a 6-mesh size, less than a 7-mesh size, less than an 8-mesh size, less than a 10-mesh size, less than a 12-mesh size, less than a 14-mesh size, less than a 16-mesh size, less than a 18-mesh size, less than a 20-mesh size, less than a 25-mesh size, less than a 30-mesh size, less than a 35-mesh size, less than a 40-mesh size, less than a 45-mesh size, less than a 50-mesh size, less than a 60-mesh size, less than a 70-mesh size, or less than an 80-mesh size).

In some cases, the dietary supplement composition provided herein contains particles having a dimension between a 5-mesh size and a 100-mesh size (e.g., between a 10-mesh size and a 80-mesh size, between a 14-mesh size and a 60-mesh size, between a 16-mesh size and a 40-mesh size, between a 20-mesh size and a 35-mesh size, between a 25-mesh size and a 30-mesh size, between a 5-mesh size and a 80-mesh size, between a 10-mesh size and a 60-mesh size, between a 12-mesh size and a 50-mesh size, between a 14-mesh size and a 40-mesh size, between a 16-mesh size and a 30-mesh size, between a 20-mesh size and a 80-mesh size, or between a 10-mesh size and a 60-mesh size).

In some cases, the dietary supplement compositions described herein can have a bulk density appropriate for providing a suitable amount of active ingredients within a specific volume (e.g., active ingredients within a capsule). In some cases, the composition can have a bulk density of at least about 200 mg/ml (e.g., at least about 300 mg/ml, at least about 400 mg/ml, at least about 500 mg/ml, at least about 600 mg/ml, at least about 700 mg/ml, at least about 800 mg/ml, at least about 800 mg/ml, at least about 900 mg/ml, at least about 1,000 mg/ml, at least about 1,100 mg/ml, at least about 1,200 mg/ml, at least about 1,300 mg/ml, at least about 1,400 mg/ml, at least about 1,500 mg/ml, or at least about 2,000 mg/ml). In some cases, the composition can have a bulk density of about 200 mg/ml to about 2,000 mg/ml (e.g., about 400 mg/ml to about 1,500 mg/ml, about 500 mg/ml to about 1,000 mg/ml, about 600 mg/ml to about 900 mg/ml, about 700 mg/ml to about 800 mg/ml, about 200 mg/ml to about 1,000 mg/ml, about 300 mg/ml to about 800 mg/ml, about 400 mg/ml to about 700 mg/ml, about 500 mg/ml to about 1,000 mg/ml, about 600 mg/ml to about 900 mg/ml, about 700 mg/ml to about 800 mg/ml, about 200 mg/ml to about 800 mg/ml, about 700 mg/ml to about 1,000 mg/ml, or about 700 mg/ml to about 2,000 mg/ml).

In some cases, the dietary supplement compositions provided herein can include coated particles (e.g., coated granulates). In some cases, the composition (e.g., in the form of tablets, gel capsules, or a powder) can include a coating material. In particular, in some embodiments, the composition can include particles coated with a material that is a “self-coating” material, which is a coating material that is made of the same ingredients as the object (e.g., particles) being coated. In other words, in some embodiments, a coating material used in the presently described materials and methods is made from the extract powder itself, such that the coating material and the granulates are both made of the same extract powder. In some cases, the coating material and the particles of the final composition are made of the exact same ingredients. Thus, in some embodiments, a coating material can be a Ganoderma-based coating material that does not include non-Ganoderma-based coating material, such as any additives, plasticizers and/or pigments. Without being bound by theory, it is believed that “self-coating” can alter the hydroscopic properties of the particles, because the crystallinity of the outer (coated) surface is modified through the application of the self-coating material.

As such, certain embodiments of the dietary supplement composition provided herein can include coated granulates. In some cases, a composition can include about 100 mg to about 600 mg of uncoated granulates, and about 1 mg to about 50 mg of a coating material applied over the granulates. The coating material can serve as a moisture-barrier between the atmosphere and the uncoated granulate to minimize, prevent, or decrease moisture absorption over time. For example, in some cases, the coating material is a thin film. In some cases, the coating material encapsulates the granulates. The coating material can fully or partially cover the granulates.

In some cases, a dietary supplement composition provided herein contains only ingredients derived from Ganoderma lucidum. In some cases, a dietary supplement composition provided herein can contain ingredients that are derived from Ganoderma lucidum and do not contain any excipients, auxiliary components, binding agents, fillers, lubricants, disintegrants, or wetting agents. For example, in some cases, the uncoated granulate, the coating material, or both, are made of only ingredients derived from Ganoderma lucidum. In some embodiments, the dietary supplement composition provided herein includes uncoated granulates, a coating material, or both, that are made of only ingredients derived from a Ganoderma lucidum extract.

In some cases, the dietary supplement compositions provided herein can be in the form of a capsule or tablet configured to have a unit dosage about equal to the daily desired dosage for a particular mammal. For example, if a mammal is to be administered 100 mg of a particular agent, each tablet can include about 100 mg in weight of that agent. As used herein, the term “mammals” generally refers to humans, but the term also can include domesticated mammals (e.g., dogs, cats, and livestock such as cows, horses, pigs, or sheep). The dosage of a particular dietary supplement composition provided herein will depend on many factors, including the general health of a mammal. In some cases, a total daily dose may be prepared for administration in the form of one or more dosage forms (e.g., two tablets or capsules, three tablets or capsules, four tablets or capsules, five tablets or capsules, or six tablets or capsules).

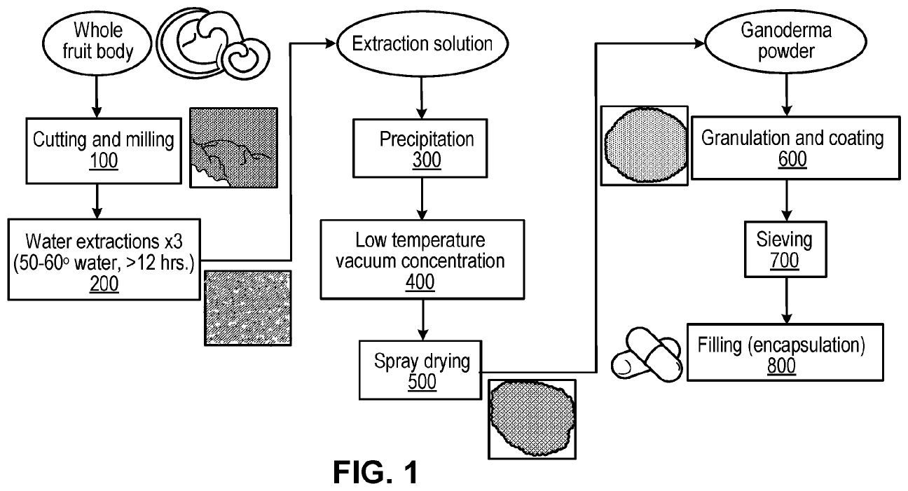

Any of the dietary supplement compositions provided herein can be made using the methods described herein. FIG. 1 shows an exemplary process for producing the dietary supplement compositions described herein.

In general, a composition provided herein can be made by: (1) obtaining or preparing a Ganoderma lucidum extract; (2) granulating the Ganoderma lucidum extract; (3) making a self-coating material solution, and (4) coating the granulates with the self-coating material solution. These four steps are described in detail below.

In Step 1, a Ganoderma lucidum raw material can be pulverized and soaked in pure water at a temperature of 60° C. or less. This can yield a clear solution. The clear solution can be optionally aspirated, concentrated under reduced pressure, and then subjected to spray drying to form an extract powder. In some embodiments, an extract powder can be commercially obtained.

In Step 2, the extract powder can be subjected to a granulation process. In some cases, the extract powder can be made into larger pieces, such as granulates or flakes, by dry granulation. In some cases, the extract powder can be made without the addition of any excipients. For example, granulation can be carried out using a dry granulation process in which granules are formed without the aid of any liquid solution. A dry granulation process can be particularly useful if the ingredient(s) to be granulated are sensitive to moisture or heat. In some cases, compaction can be applied during granulation to densify an extract powder, and/or milling can be used to form granules. The screen size and speed used during the milling can determine the particle size. In some cases, a dry granulation process can be carried out using a slugging tool, a roller compactor, or a tablet press machine.

Dry granulation can produce granulates having a desired particle size (e.g., 16 to 40 mesh-sized particles, or about 0.0469 inch to about 0.0165 inch) that are larger in size than the particles of an extract powder obtained from spray drying. Because granulation can be achieved without any additives, it is possible to carry out the granulation process using only ingredients derived from Ganoderma lucidum.

In Step 3, an amount of the extract powder can be used to produce a self-coating material. To produce a self-coating material, a Ganoderma lucidum extract powder can be completely dissolved in pure water, formulated into a coating solution of a desired proportion, and then applied to the granulates to form a “self-coated” Ganoderma lucidum extract. The “self-coated” Ganoderma lucidum extract described herein differs from other extracts in which a coating material or coating process is carried out by using other materials such that they contain no “self-coated” Ganoderma lucidum extract.

In some embodiments, a suitable amount of an extract powder, as the solute of the coating material agent, is completely dissolved in water (e.g., pure water or distilled water) to prepare a desired proportion of the coating solution, which then can be applied to the granulates. In some embodiments, a coating solution can contain from about 1% w/w to about 50% w/w of a Ganoderma lucidum extract.

In Step 4, the above-described granulates (see Step 2) can be coated with the above-described self-coating solution (see Step 3). In some embodiments, the coating solution can be applied to the granulates using, for example, a pump (e.g., a peristaltic pump) and/or by spraying. In some embodiments, the coating step can be carried out by placing the granulates or flakes in a coating pot, preheating the flakes to an appropriate (e.g., elevated) temperature, turning on the spray gun, and optionally adjusting the spray gun pressure and spray volume to a desired setting. For example, in some embodiments, the particles can be placed in a coating pan and heated before and/or during the coating process. In some embodiments, a spray gun can be employed in which a pressure and spray volume of the spray gun are adjusted to an appropriate level such that the coating solution is evenly sprayed on the granulates. A coating film can be formed by contacting and spreading the coating solution on the particles, such that a coating film forms on the particles upon contact with the coating solution and the solvent (e.g., water) evaporates. In some cases, the coating solution can be evenly sprayed on a rotating sheet, and through contact and spreading, the droplets can be combined with each other and the solvent can be volatilized to form a coating film over the granulates.

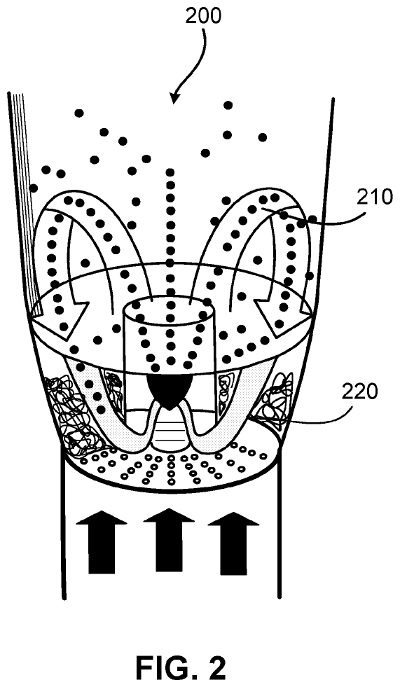

In some cases, a coating material can be applied to granulates using a spray fluidized bed. FIG. 2 illustrates an exemplary spray fluidized bed (200) that can be used for coating particles. In some cases (e.g., as depicted in FIG. 2), a spray fluidized bed can be connected (as a bottom portion) to dry granulating equipment, and the coating process can be accomplished during air-based suspension of the granulates. In some embodiments, a dry-spray extract powder can be formed into granulates and simultaneously coated, thus, producing coated granulates. For example, as depicted in step (210), a coating solution can be sprayed out from a nozzle and mixed with fluidized particles complete the coating process. The particles (220) aggregated at the bottom portion of the spray fluidized bed depicted in FIG. 2 are the particles to be coated.

As depicted in FIG. 1, an exemplary process for producing the dietary supplement compositions provided herein can include:
obtaining whole fruit bodies of Ganoderma lucidum,
reducing the size of the fruit bodies by, for example, cutting and milling (100) the fruit bodies,
obtaining an extract powder by:
   applying a water extraction process (200) to the fruit bodies that introduces an extraction solvent (e.g., water) to obtain an extraction solution;
   precipitating (300) the extraction solution;
   optionally applying a low temperature and vacuum condition (400) to form a concentrate solution;
spray drying (500) the extract solution or concentrate solution to produce a Ganoderma lucidum powder;
granulating (600) the powder to produce granulates and optionally coating the granulates;
optionally sieving (700) the granulates; and
and optionally filling (800) capsules (i.e., capsulation).

In some cases, in the obtaining step, the raw material (e.g., Ganoderma fruit) may be grown and subsequently harvested. In some cases, the Ganoderma lucidum components can be subjected to preconditioning following harvesting, such as being subjected to a drying step.

In some cases, reducing the size of the Ganoderma lucidum raw material can be achieved using means that are not limited to only cutting and milling. For example, the raw material can be crushed, for example, using a grinder.

The extract powder can be extracted using any suitable method. For example, the extraction process can use a solvent, such as water, an alcohol, or both. With water extraction, the main active ingredient that can be obtained from Ganoderma lucidum typically is polysaccharide. In some cases, the extraction process can include one extraction or more than one extraction (e.g., 2, 3, 4, 5, or more than 5 extractions). In some cases, the raw material can be soaked in pure water at a temperature below 60° C. to absorb the clear liquid, and then subjected to a reduced pressure before, during, or after extraction but prior to spray drying to obtain an extract powder. Ganoderma lucidum extracts can also be made using various processes described elsewhere (e.g., Chinese Patent No. CN1813820A).

During capsulation, empty capsules may be filled with either the granules or coated granulates, and then packaged into finished products.

In some cases, the compositions provided herein containing coated particles can have a moisture content that is reduced as compared to the moisture content of a corresponding composition containing particles that are not coated. In some cases, for example, the moisture content of a dry granulated and coated Ganoderma lucidum composition provided herein can be at least 5% lower (e.g., at least 7%, at least 10%, at least 15%, at least 20%, at least 25%, at least 30%, at least 35%, at least 40%, at least 45%, or at least 50% lower) than the moisture content of a corresponding dry granulated Ganoderma lucidum composition that lacks the coating. For example, for a dry granulated and coated composition with a moisture content of 2.5%, the moisture content is 16.7% lower than that of a dry granulated, non-coated composition having a moisture content of 3%. Any appropriate method can be used to determine the moisture content of a composition, including, for example, using a moisture meter as described in the Examples herein.

In some cases, the compositions provided herein can contain coated particles having a porosity that is reduced as compared to the porosity of corresponding particles that are not coated. In some cases, for example, the porosity of dry granulated and coated Ganoderma lucidum particles can be at least 5% lower (e.g., at least 7%, at least 10%, at least 15%, at least 20%, at least 25%, at least 30%, at least 35%, at least 40%, at least 45%, or at least 50% lower) than the porosity of corresponding dry granulated Ganoderma lucidum particles that lack the coating. Any appropriate method can be used to determine the degree of porosity of particles in a composition provided herein, including methods that measure the rate of caking, for example.

While the above-described embodiments relate to compositions containing only Ganoderma lucidum-derived ingredients, it is to be noted that in some embodiments, the compositions provided herein may contain other ingredients in addition to those derived from Ganoderma lucidum. For example, in certain embodiments, the dietary supplement compositions provided herein can be formulated for oral administration and can include suitable excipients, flavorings, colorants, and/or other ingredients. For oral administration, tablets or capsules can be prepared with pharmaceutically acceptable excipients such as binding agents, fillers, lubricants, disintegrants, and/or wetting agents. In some cases, a dietary supplement composition provided herein can include one or more of the following ingredients in addition to the Ganoderma lucidum component: carotenoids (e.g., alpha carotenoids), phenolic compounds (e.g., flavonoids, flavonols, flavanones, catechins, anthocyanins, isoflavones, dihydroflavonols, and/or chalcones), phenolic acids (e.g., ellagic acid, tannic acid, and/or vanillin), hydroxycinnamic acid derivatives (e.g., caffeic acid, chlorogenic acid, ferulic acid, curcumin, and/or coumarins), lignans, allyl sulphides from onion or garlic, and/or essential oils (e.g., melaleuca oil, clove oil, cinnamon bark oil, thyme oil, oregano oil, mountain savory oil, cistus oil, eucalyptus globulus oil, orange oil, lemongrass oil, helichrysum oil, ravensara oil, lemon oil, spearmint oil, and/or lavender oil). In some cases, a dietary supplement composition provided herein can include one or more of Vitamin E, tocopherol, tocotrienol, Vitamin A, carotene, lutein, astaxanthin, CoQ10, Vitamin C, folate, uric acid, Vitamin B12, and folic acid.

In some cases, dietary supplement compositions provided herein can contain a pharmaceutically acceptable carrier appropriate for administration to a mammal. Suitable pharmaceutically acceptable carriers include, without limitation, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Pharmaceutically acceptable carriers also can include physiologically acceptable aqueous vehicles (e.g., physiological saline) or other carriers appropriate for oral administration.

The invention will be further described in the following examples, which do not limit the scope of the invention described in the claims.

### EXAMPLES

### Example 1- Exemplary Dietary Supplement Composition

An exemplary dietary supplement composition was made as described below.

A Ganoderma lucidum extract powder obtained using standard techniques was subjected to dry granulation by roller compaction (see, e.g., U.S. Pat. No. 7,998,505) to yield 600 g of particles (16 to 40 mesh, or about 0.0469 inch to about 0.0165 inch). In addition, 12 g of Ganoderma lucidum extract powder were added to 138 g of pure water, and stirred and dissolved completely to obtain a coating material for later use. The 600 g of granulates (16 to 40 mesh, or about 0.0469 inch to about 0.0165 inch) were placed in the coating pan of a bottom-spray fluidized bed, the air inlet volume was set to 50 m³/h, and the particles were preheated to 35 to 45° C. The above-mentioned coating material was sprayed onto the particles via a feeding pump at a speed of 5 ml/min. Semi-finished products were prepared after completion of the coating solution spraying, and were used to fill hard capsules to obtain finished products.

### Example 2- Exemplary Dietary Supplement Composition

An exemplary dietary supplement composition was made as described below.

A Ganoderma lucidum extract powder was subjected to dry granulation by roller compaction (see, e.g., U.S. Pat. No. 7,998,505) to yield 600 g of particles (16 to 40 mesh, or about 0.0469 inch to about 0.0165 inch). In addition, 30 g of Ganoderma lucidum extract powder were added to 220 g of pure water, stirred, and dissolved completely to obtain a coating material for later use. The 600 g of particles (16 to 40 mesh, or about 0.0469 inch to about 0.0165 inch) were placed in the coating pan of a bottom-spray fluidized bed, the air inlet volume was set to 55 m³/h, and the particles were preheated to 35 to 45° C. The above-mentioned coating material was sprayed onto the particles via a feeding pump at a speed of 4 ml/min. Semi-finished products that resulted after completion of the coating solution spraying were used to fill hard capsules to obtain finished products.

### Example 3- Dietary Supplement Compositions

Exemplary dietary supplement compositions (Samples 2 & 3) along with a control supplement composition (Sample 1) were prepared. Sample 1 was obtained using standard techniques. Sample 2 was prepared by dry granulation as described in Examples 1 and 2, and Sample 3 was prepared by dry granulation and coating as described in Examples 1 and 2). Each sample is listed in Table 1 below.

TABLE 1
Dietary Supplement Compositions

| Sample No. | Sample Description | Composition |
|---|---|---|
| 1 | Spray Drying (Control) | 100% Ganoderma lucidum |
| | | extract* |
| 2 | Dry Granulation | 100% Ganoderma lucidum |
| | | extract* |
| 3 | Coated Dry Granulation | 100% Ganoderma lucidum |
| | | extract* |

*No additional ingredients were added during any of the processing methods used to make the samples.

Sample 1, a control sample, was a pure Ganoderma lucidum powder that was made from spray drying an extract solution. Sample 2 was an exemplary composition that was made by applying a dry granulation process to a pure Ganoderma lucidum powder. Sample 3 was an exemplary composition that was made by applying dry granulation and coating processes to a pure Ganoderma lucidum powder.

Each sample was evaluated for moisture content, fill weight, production efficiency, and stability, as discussed below.

### I. Moisture Content

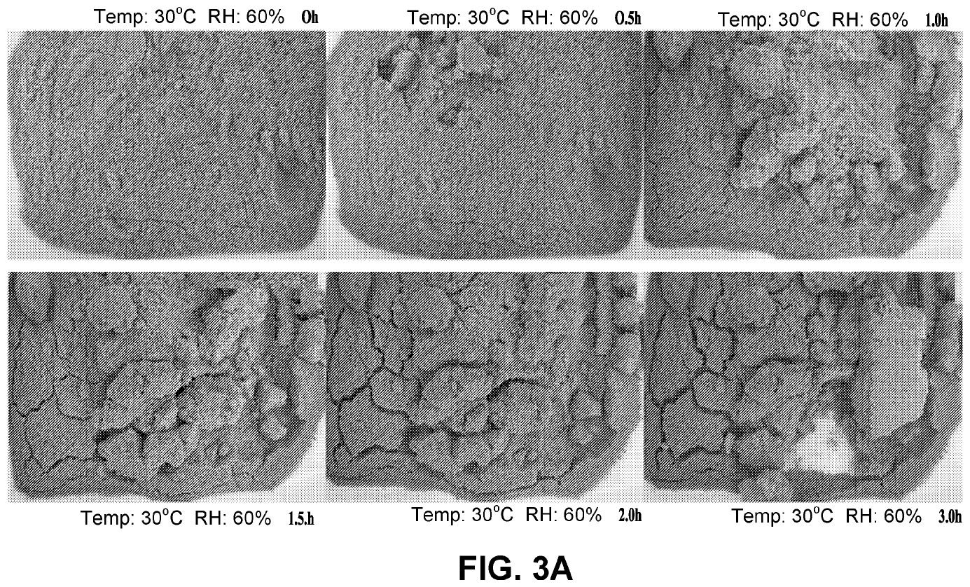

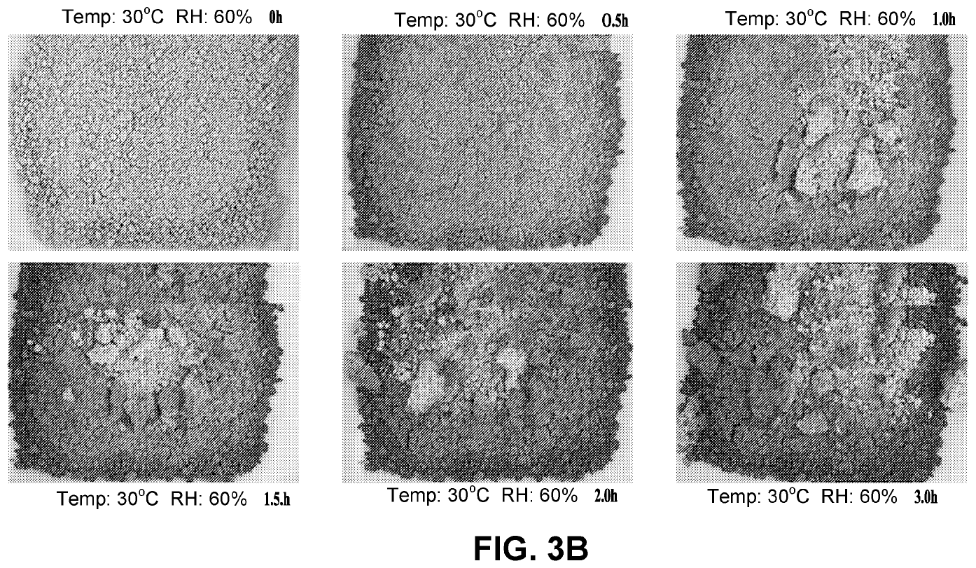

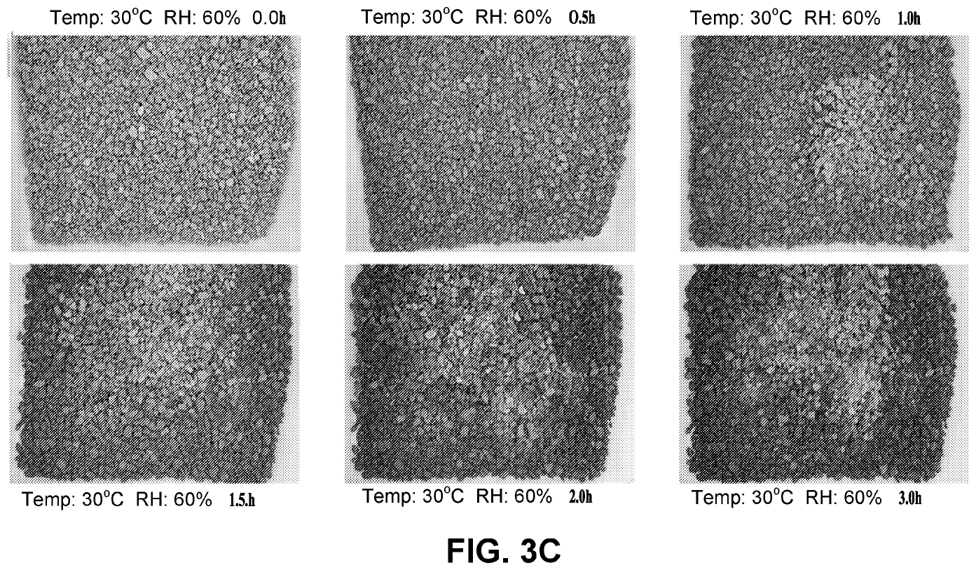

The samples were subjected to a constant temperature of 30° C. and humidity of RH 60%, and the appearance of each sample was observed over time. As shown in FIGS. 3A-3C, photos of the samples were taken at various times (0.0 h, 0.5 h, 1.0 h, 1.5 h, 2.0 h, 3.0 h). An initial weight of 5.0 g of each sample was re-measured at various times (0.0 h, 0.5 h, 1.0 h, 1.5 h, 2.0 h, and 3.0 h). The results showed that while all of the samples appeared hygroscopic and discolored, the spray drying powder (Sample 1; FIG. 3A) exhibited the most caked appearance over time. No caking occurred in the coated sample (Sample 3; FIG. 3C), while the non-coated, dry granulated sample had an intermediate appearance (FIG. 3B).

The moisture content of each sample was measured with a moisture meter at various times, as shown in Table 2.

TABLE 2
Moisture Content Data

| Time (h) | 0.0 | 0.5 | 1.0 | 1.5 | 2.0 | 3.0 |
|---|---|---|---|---|---|---|
| Spray drying | 3.21% | 5.32% | 6.36% | 6.73% | 7.50% | 7.99% |
| Dry | 3.40% | 4.05% | 4.32% | 4.69% | 4.75% | 6.59% |
| Granulation | | | | | | |
| Coating | 3.60% | 3.72% | 3.80% | 3.85% | 4.71% | 5.57% |

The results showed that moisture increased over time in all three samples, but the spray dried sample (Sample 1) had the highest moisture content while the coated product (Sample 3) had the least.

### II. Filling Weight

The samples were filled into ten hard-shell capsules and weighed, as shown in Table 3A. The filling weight of each capsule also was determined (Table 3B).

TABLE 3A
Capsule Weight Data

| | Empty | Spray dried | Granulated | Coated |
|---|---|---|---|---|
| | Capsules | Sample | Sample | Sample |
| Weight | 0.97 g | 3.09 g | 6.17 g | 6.20 g |
| (10 capsules) | | | | |

TABLE 3B
Filling Weight Data

| | Spray drying | Granulation | Coating |
|---|---|---|---|
| | Sample | Sample | Sample |
| Filling weight of each capsule | 212 mg | 520 mg | 523 mg |

The results showed that granulation of the Ganoderma extract powder, as well as granulation and coating of the Ganoderma extract powder, significantly increased the filling weight of a capsulated product.

### III. Stability Testing

The samples were subjected to accelerated aging conditions of 37° C., RH 75%, and the moisture content of each sample was measured with a moisture meter at various time points (Table 4).

In addition, moisture content changes of the samples under ambient temperature conditions were measured, with samples under refrigeration conditions (5° C., RH 60%) used as controls, as shown in Table 5.

TABLE 4
Stability Data

| | | | Dry granulation |
|---|---|---|---|
| | Spray drying | Dry granulation | and coating |
| Shelf-life | Sample 1 | Sample 2 | Sample 3 |
| (in months) | (moisture content) | (moisture content) | (moisture content) |
| 0 | 3.21% | 2.40% | 2.40% |
| 1 | 5.49% | 4.39% | 4.20% |
| 1.5 | 5.32% | 4.60% | 4.41% |
| 2 | 5.60% | 4.80% | 4.40% |
| 2.5 | 6.32% | 5.22% | 4.40% |
| 3 | 6.69% | 5.60% | 4.00% |
| 4 | / | 5.65% | 4.20% |
| 5 | / | 5.98% | 4.75% |
| 6 | / | / | 5.01% |

Note:
“/” indicates that the sample was in an agglomerated state and the moisture content detection was incomplete.

TABLE 5
Stability Data in Ambient Conditions

| Ambient Shelf- | Dry granulation Sample | | Coating Sample | |
|---|---|---|---|---|
| life (in months) | (moisture content) | | (moisture content) | |
| Conditions | 5° C., RH 60% | Ambient | 5° C., RH 60% | Ambient |
| 0 | 2.40% | 2.40% | 2.40% | 2.40% |
| 6 | 3.41% | 3.65% | 3.22% | 3.46% |
| 12 | 4.22% | 4.03% | 3.53% | 3.98% |
| 16 | 4.92% | 4.15% | 3.76% | 4.09% |

### IV. Production Efficiency

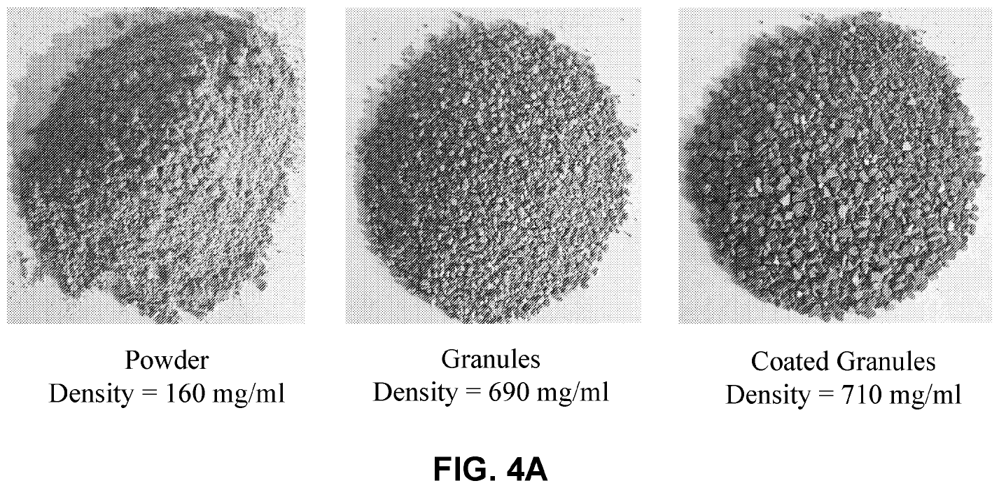

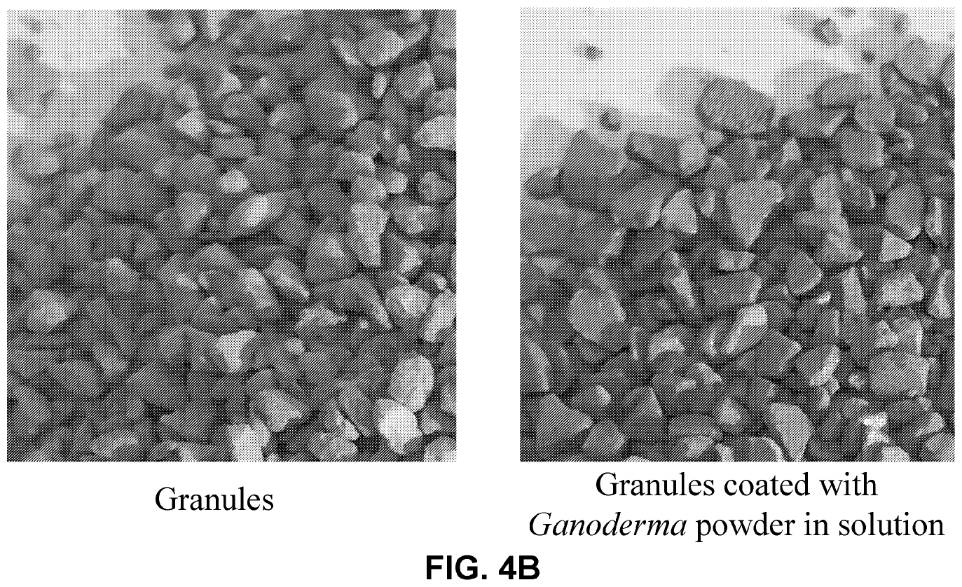

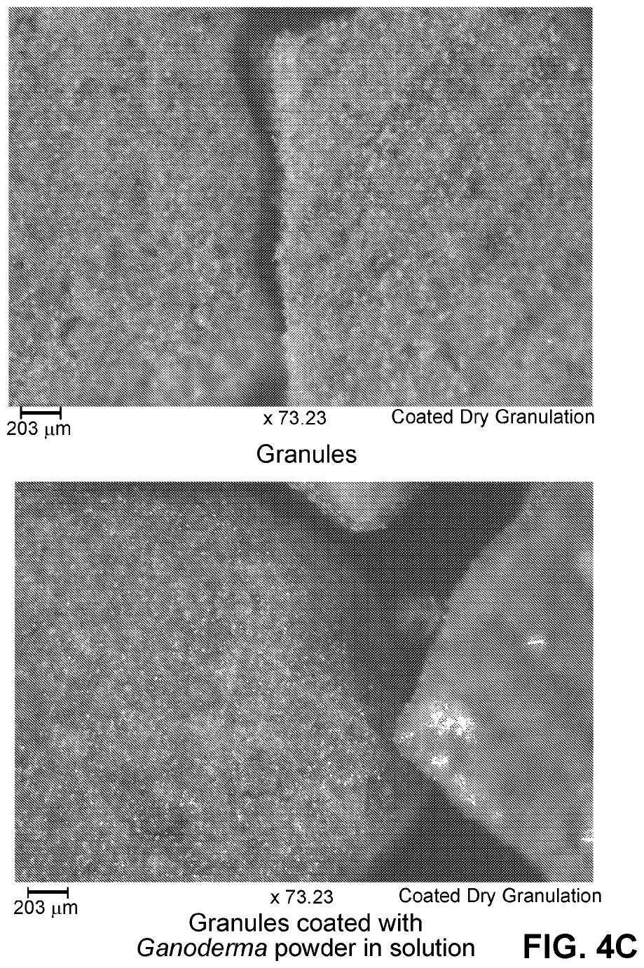

The samples were subjected to various manufacturing processes. The dry sprayed sample was subjected to 5 minutes of continuous encapsulation and 1.5 hours of cleaning, with about 10% powder loss in filling. The dry granulated sample was subjected to 15 minutes of continuous encapsulation and 2 minutes cleaning, with about 3% powder loss in filling. The dry granulated and coated sample was subjected to 1.5 hours of continuous encapsulation and 2 minutes of cleaning, with about 0.5% powder loss in filling. Images showing the three samples are provided in FIG. 4A. The bulk densities and filling loss ratios of the three test samples are provided in Table 6.

TABLE 6
Processing Time Data

| Process | Bulk Density | Filling loss ratio |
|---|---|---|
| Dry spraying | 160 mg/ml | 10% |
| Dry Granulation | 690 mg/ml | 3% |
| Dry Granulation and Coating | 710 mg/ml | 0.5% |

During the process of filling capsules, small sample particles (e.g., powder on the surface of individual particles) can adhere to the discharge port and/or the disc of the filling machine. As a result, some particles may be lost during the filling process. The filling loss ratio is defined by the weight of the material lost during the filling process. As indicated in Table 6, the dry sprayed particles exhibited a 10% loss ratio, the dry granulated particles exhibited a 3% loss ratio, and the dry granulated and coated particles exhibited only a 0.5% filling loss ratio. The relative level of powder on the surface of dry granulated vs. dry granulated and coated particles is shown in FIGS. 4B and 4C. The surface of the dry granulated particles appeared relatively rough due to the presence of powder, while the surface of the dry granulated and coated particles appeared relatively smooth.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of making a composition, the method comprising:
granulating a powdered first sample of Ganoderma lucidum extract to form granulates; and
applying a coating material on the granulates, the coating material comprising a second sample of Ganoderma lucidum extract;
wherein the composition is substantially free of non-Ganoderma lucidum derived materials.

2. The method of claim 1, wherein the granulating is performed without using non-Ganoderma lucidum derived materials.

3. The method of claim 1, wherein the applying of the coating material is performed without using non-Ganoderma lucidum derived materials.

4. The method of claim 1, wherein the granulates, the coating, or the granulates and the coating are substantially free of auxiliary excipients or additives.

5. (canceled)

6. The method of claim 1, wherein the granulating step comprises dry granulating the powder.

7. The method of claim 1, wherein the applying step comprises applying a solution comprising a solute and a solvent to the granulates.

8. The method of claim 7, wherein the solute is the second sample of Ganoderma lucidum extract.

9. The method of claim 8, wherein the solvent is water.

10. The method of claim 10, wherein the solution comprises about 1% w/w to about 40% w/w of the second sample of Ganoderma lucidum extract.

11. The method of claim 1, wherein:
a) the granulates have a dimension ranging between a 16 mesh size and a 100 mesh size;
b) the granulates have a size between 0.0469 inch and 0.0059 inch,
c) the composition has a bulk density of about 250 mg/ml to about 2000 mg/ml, or
d) any combination thereof.

12-13. (canceled)

14. The method of claim 1, wherein the applying step comprises using a spray fluidized bed.

15. The method of claim 1, wherein the powder was obtained by subjecting Ganoderma lucidum to water extraction and then spray drying.

16. The method of claim 1, wherein the composition has a moisture content that is reduced compared to the moisture content of a corresponding composition that lacks the coating material.

17. The method of claim 1, wherein the coated granulates have a porosity that is reduced compared to the porosity of corresponding uncoated granulates.

18. A composition made using the method of claim 1.

19. A health care product, which comprises, as an active ingredient, the composition of claim 18.

20. The health care product of claim 19, wherein the health care product is a capsulated product

21. A method of making a composition, comprising:
granulating a powder comprising one or more extracts to form granulates; and
applying a coating material on the granulates, the coating material comprising the one or more extracts;
wherein the composition is substantially free of materials that are not derived from the one or more extracts.

22. The method of claim 21, wherein the one or more extracts each have a bulk density less than 200 mg/ml, wherein the composition has a bulk density of about 250 mg/ml from about 2000 mg/ml, or wherein the one or more extracts each have a bulk density less than 200 mg/ml and composition has a bulk density of about 250 mg/ml from about 2000 mg/ml.

23-24. (canceled)

25. A method of making a composition, the method comprising:
granulating a powdered first sample of Ganoderma extract to form granulates; and
applying a coating material on the granulates, the coating material comprising a second sample of Ganoderma extract;
wherein the composition is substantially free of non-Ganoderma derived materials.
